(19) United States
(12) Patent Application Publication
(10) Pub. No.: US 2019/0313951 A1
(43) Pub. Date: Oct. 17, 2019

(54) ORGANISM ANALYZING APPARATUS AND ORGANISM ANALYZING METHOD

(71) Applicant: SEIKO EPSON CORPORATION, Tokyo (JP)

(72) Inventor: Jun TAKEUCHI, Matsumoto-shi (JP)

(73) Assignee: SEIKO EPSON CORPORATION, Tokyo (JP)

(21) Appl. No.: 16/382,253

(22) Filed: Apr. 12, 2019

(30) Foreign Application Priority Data
Apr. 13, 2018 (JP) 2018-077876

(51) Int. Cl. A61B 5/145; A61B 5/00; A61B 5/11; A61B 5/0205; A61B 5/08

(52) U.S. Cl. CPC A61B 5/14532; A61B 5/4845; A61B 5/024; A61B 5/0205; A61B 5/082; A61B 5/1118

(57) ABSTRACT

An organism analyzing apparatus includes an information acquiring section configured to acquire an amount of change in a blood glucose level due to ingestion of an organism and a sugar-content estimating section configured to estimate, according to the amount of change in the blood glucose level, sugar content ingested by the organism through the ingestion.

The present application is based on, and claims priority from Japanese Application Serial Number 2018-077876, filed Apr. 13, 2018, the disclosure of which is hereby incorporated by reference herein in its entirety.

### BACKGROUND

### 1. Technical Field

The present disclosure relates to a technique for estimating sugar content ingested by an organism.

### 2. Related Art

Management of a blood glucose level and sugar content is important for treatment or prevention of lifestyle related diseases such as diabetes. JP-A-2012-27758 (Patent Literature 1) discloses a technique for predicting a blood glucose level of a user according to an amount of meal and meal content input by the user.

It is necessary to accurately grasp sugar content in order to appropriately manage sugar content ingested by the user. However, work for inputting the amount of meal and the meal content is a heavy burden for the user.

### SUMMARY

An organism analyzing apparatus according to an aspect of the present disclosure includes: an information acquiring section configured to acquire an amount of change in a blood glucose level due to ingestion of an organism; and a sugar-content estimating section configured to estimate, according to the amount of change in the blood glucose level, sugar content ingested by the organism through the ingestion.

An organism analyzing method according to another aspect of the present disclosure includes: acquiring an amount of change in a blood glucose level due to ingestion of an organism; and estimating, according to the amount of change in the blood glucose level, sugar content ingested by the organism through the ingestion.

### BRIEF DESCRIPTION OF THE DRAWINGS

FIG. 1 is a block diagram illustrating the configuration of an organism analyzing apparatus in a first embodiment.

FIG. 2 is a graph showing a temporal change in a blood glucose level after ingestion.

FIG. 3 is a graph showing a relation between an amount of increase of a blood glucose level and ingested sugar content after ingestion.

FIG. 4 is an explanatory diagram of reference information.

FIG. 5 is a graph showing a time sequence of a blood glucose level.

FIG. 6 is a flowchart illustrating a specific procedure of sugar content estimation processing for estimating ingested sugar content.

FIG. 7 is a graph showing temporal changes in a blood glucose level and a pulse rate at the time when a rest state is maintained immediately after ingestion.

FIG. 8 is a graph showing temporal changes of a blood glucose level and a pulse rate at the time when a subject exercises immediately after ingestion.

FIG. 9 is a block diagram illustrating the configuration of an organism analyzing apparatus in a second embodiment.

FIG. 10 is a flowchart illustrating a specific procedure of processing in which a sugar-content estimating section in the second embodiment estimates ingested sugar content.

FIG. 11 is a graph showing temporal changes of a blood glucose level and a pulse rate at the time when a subject ingests food without drinking alcohol.

FIG. 12 is a graph showing temporal changes of a blood glucose level and a pulse rate at the time when the subject ingests food while drinking alcohol.

FIG. 13 is a block diagram illustrating the configuration of an organism analyzing apparatus in a third embodiment.

FIG. 14 is a flowchart illustrating a specific procedure of processing in which a sugar-content estimating section in the third embodiment estimates ingested sugar content.

FIG. 15 is a block diagram illustrating the configuration of an organism analyzing apparatus in a modification.

FIG. 16 is a block diagram illustrating the configuration of an organism analyzing apparatus in a modification.

### DESCRIPTION OF EXEMPLARY EMBODIMENTS

### First Embodiment

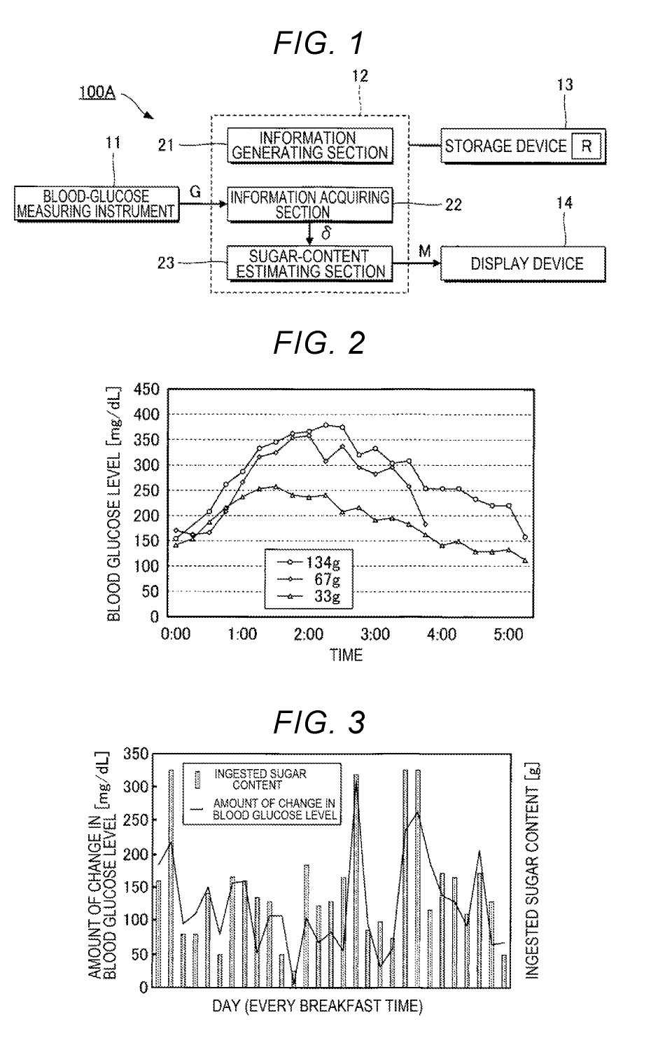

FIG. 1 is a block diagram illustrating the configuration of an organism analyzing apparatus 100A according to a first embodiment of the present disclosure. The organism analyzing apparatus 100A is a measuring apparatus estimating sugar content ingested by a subject (an example of an organism) (hereinafter referred to as “ingested sugar content”). As illustrated in FIG. 1, the organism analyzing apparatus 100A includes a blood-glucose measuring instrument 11, a control device 12, a storage device 13, and a display device 14. The blood-glucose measuring instrument 11 separate from the organism analyzing apparatus 100A may be connected to the organism analyzing apparatus 100A by wire or radio. That is, the blood-glucose measuring instrument 11 is omitted from the organism analyzing apparatus 100A.

The blood-glucose measuring instrument 11 measures a blood glucose level G of the subject. The blood glucose level G is the concentration of glucose present in blood of the subject. The measurement of the blood glucose level G by the blood-glucose measuring instrument 11 is repeated, for example, at a predetermined cycle. A publicly-known technique is optionally adopted for the measurement of the blood glucose level G. For example, SMBG (Self Monitoring of Blood Glucose), CGM (Continuous Glucose Monitoring), FGM (Flash Glucose Monitoring), or any technique for measuring the blood glucose level G in a noninvasive manner is used for the measurement of the blood glucose level G by the blood-glucose measuring instrument 11. The blood glucose level G is typically an absolute value meaning weight per unit volume (mg/dL) but may be a relative value to a predetermined reference value. A numerical value obtained by smoothing a measurement value by the blood-glucose measuring device 11 on a time axis may be used as the blood glucose level G.

FIG. 2 is a graph showing a temporal change in a blood glucose level after ingestion of polished rice. In FIG. 2, temporal changes of blood glucose levels are also shown concerning a respective plurality of cases in which ingested sugar content is differentiated (33 g, 67 g, and 134 g). As it is understood from FIG. 2, the blood glucose level rises overtime through ingestion of sugar. A peak value of the blood glucose level depends on the ingested sugar content. Specifically, the peak value of the blood glucose level is a higher numerical value as the ingested sugar content increases.

FIG. 3 is a graph showing, day by day, a relation between an amount of change (an amount of increase) of a blood glucose level and ingested sugar content after ingestion. A bar graph represents ingested sugar content (g). Aline graph represents, as an amount of change in a blood glucose level, an increase width (mg/dL) of a blood glucose level obtained by comparing a blood glucose level before meal and a blood glucose level two hours after the meal. The horizontal axis is a time axis representing a measurement day (every breakfast time). As it is understood from FIGS. 2 and 3, the amount of change in the blood glucose level and the ingested sugar content correlate with each other. That is, there is a tendency that the amount of change in the blood glucose level is larger as the ingested sugar content is larger. Based on the tendency, the organism analyzing apparatus 100A in the first embodiment estimates ingested sugar content M from an amount of change δ of the blood glucose level G measured by the blood-glucose measuring instrument 11.

The control device 12 is an arithmetic processing device such as a CPU (Central Processing Unit) or an FPGA (Field Programmable Gate Array). The control device 12 controls the entire organism analyzing apparatus 100A. It is also possible to adopt a configuration in which the function of the control device 12 is distributed to a plurality of integrated circuits or a configuration in which the function of the control device 12 is realized by a dedicated electronic circuit. The display device 14 is configured by, for example, a liquid crystal display panel. The display device 14 displays various images including an estimation result of the ingested sugar content M under the control by the control device 12.

The storage device 13 is configured by, for example, a nonvolatile semiconductor memory. The storage device 13 stores computer programs executed by the control device 12 and various data used by the control device 12. In FIG. 1, the control device 12 and the storage device 13 are illustrated as separate elements. However, the control device 12 including the storage device 13 can also be realized by, for example, an ASIC (Application Specific Integrated Circuit).

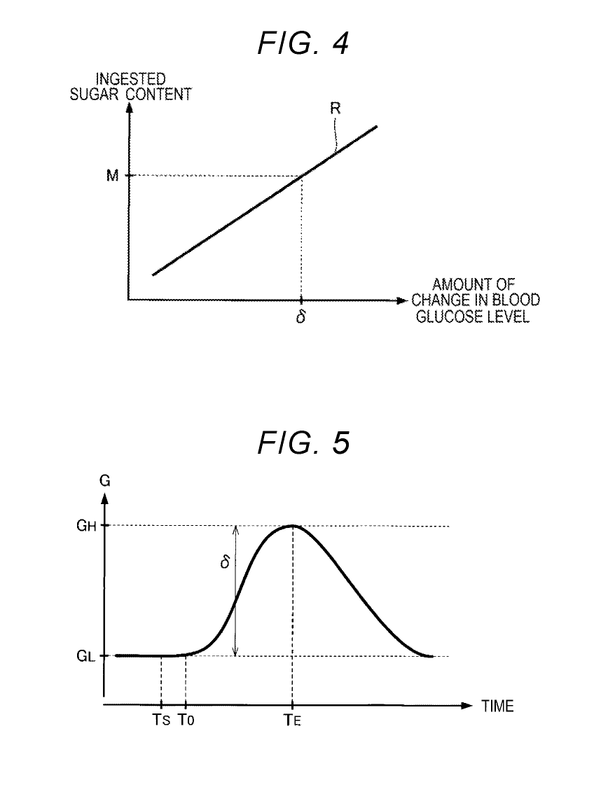

The storage device 13 in the first embodiment stores reference information R representing the relation between the amount of change δ of the blood glucose level G and the ingested sugar content M. FIG. 4 is an explanatory diagram of the reference information R. As illustrated in FIG. 4, the reference information R specifies, for example, a proportional relation between the amount of change in the blood glucose level and the ingested sugar content. The relation between the amount of change in the blood glucose level and the ingested sugar content is not limited to a linear relation illustrated in FIG. 4.

The control device 12 (an example of a computer) executes the computer programs stored in the storage device 13 to realize a plurality of functions (an information generating section 21, an information acquiring section 22, a sugar-content estimating section 23) for estimating the ingested sugar content M of the subject from a time sequence of the blood glucose level G measured by the blood-glucose measuring instrument 11. The functions of the control device 12 may be realized by a plurality of devices configured separately from one another.

The information generating section 21 generates the reference information R. Specifically, the information generating section 21 generates the reference information R according to a change in a blood glucose level of the subject when the subject ingests specific food, sugar content of which is known. That is, the information generating section 21 generates the reference information R indicating a relation between an amount of change in a blood glucose level and sugar content of the specific food when the subject ingests the specific food. For example, the reference information R is generated from a result of an oral glucose tolerance test (OGTT). The reference information R may be generated from a result obtained by measuring insulin secretion capacity or insulin resistance of the subject. The reference information R explained above is generated before the estimation of the ingested sugar content M of the subject and stored in the storage device 13. For example, the reference information R is generated during a first use of the organism analyzing apparatus 100A. The relation between the amount of change in the blood glucose level and the ingested sugar content is different according to a physical condition (e.g., a disease state of diabetes) of an individual subject. Therefore, the relation indicated by the reference information R could be different for each subject.

The information acquiring section 22 shown in FIG. 1 acquires the amount of change δ of the blood glucose level G of the subject. Specifically, the information acquiring section 22 specifies the amount of change δ from the time sequence of the blood glucose level G sequentially measured by the blood-glucose measuring instrument 11. FIG. 5 is a graph showing the time sequence of the blood glucose level G. As illustrated in FIG. 5, the information acquiring section 22 specifies the amount of change δ of the blood glucose level G based on a blood glucose level GL at a point in time TS on the time axis. As illustrated in FIG. 5, the information acquiring section 22 calculates, as the amount of change δ of the blood glucose level G, a difference value (δ=GH−GL) between the blood glucose level GL at the point in time TS and a blood glucose level GH at a point in time TE after the point in time TS.

The point in time TS is a point in time corresponding to a start of ingestion (i.e., meal) by the subject. The information acquiring section 22 in the first embodiment specifies the point in time TS from the time sequence of the blood glucose level G measured by the blood-glucose measuring instrument 11. Specifically, as illustrated in FIG. 5, the point in time TS is specified according to a point in time T0 when the blood glucose level G starts to increase. The blood glucose level G starts to increase later than the start of the ingestion by the subject. Therefore, the information acquiring section 22 in the first embodiment specifies, as the start point TS of the ingestion, a point in time a predetermined time earlier than the point in time T0 when the blood glucose level G starts to increase. The point in time T0 when the blood glucose level G starts to increase is, for example, a point in time when a rate of change (i.e., a gradient with respect to the time axis) of the blood glucose level G is larger than a predetermined threshold. When the blood glucose level G is simply measured, the point in time T0 may be regarded as the start point TS of the ingestion.

On the other hand, the point in time TE is a point in time of a peak of the blood glucose level G. That is, the blood glucose level GH at the point in time TE is a maximum value (a peak value) of the blood glucose level G. Specifically, the information acquiring section 22 specifies the point in time TE from a predetermined range including a point in time when a predetermined time elapses from the point in time TS (or the point in time T0). The predetermined time is a delay time until the blood glucose level G reaches a maximum value from the start of the ingestion by the subject. The predetermined time is measured for each subject from a result of the oral glucose tolerance test or the like.

The sugar-content estimating section 23 shown in FIG. 1 estimates the ingested sugar content M according to the amount of change δ acquired by the information acquiring section 22. The reference information R stored in the storage device 13 is used for the estimation of the ingested sugar content M. Specifically, as illustrated in FIG. 4, the sugar-content estimating section 23 specifies the ingested sugar content M corresponding to the amount of change δ of the blood glucose level G under the relation indicated by the reference information R stored in the storage device 13. The ingested sugar content M is stored in the storage device 13. The sugar-content estimating section 23 causes the display device 14 to display the ingested sugar content M.

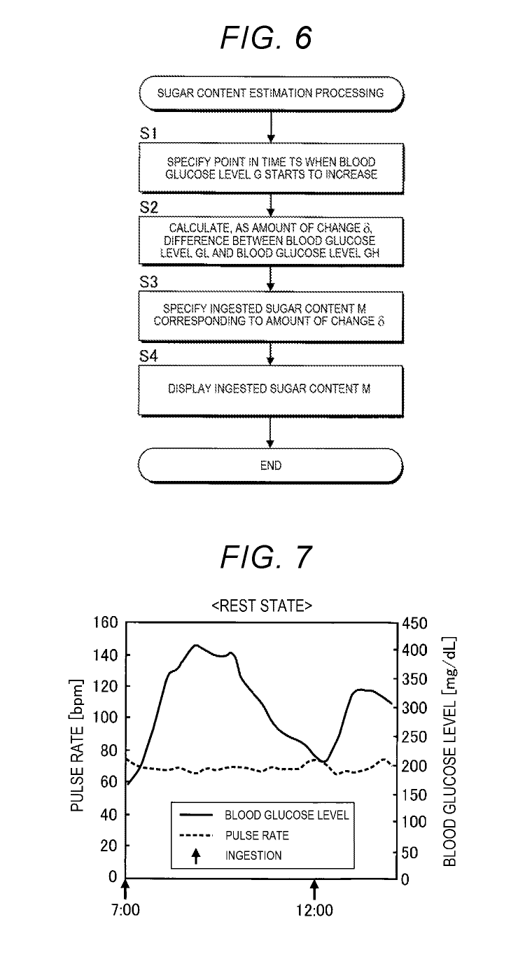

FIG. 6 is a flowchart illustrating a specific procedure of processing in which the control device 12 estimates the ingested sugar content M (hereinafter referred to as “sugar content estimation processing”). For example, the sugar content estimation processing shown in FIG. 6 is started according to an instruction from the subject. At a stage when the sugar content estimation processing is started, the reference information R is generated by the information generating section 21 and stored in the storage device 13.

When the sugar content estimation processing is started, the information acquiring section 22 specifies the amount of change δ of the blood glucose level G from the time sequence of the blood glucose level G sequentially measured by the blood-glucose measuring instrument 11 (S1 and S2). Specifically, the information acquiring section 22 specifies the start point TS of the ingestion by the subject (S1). The information acquiring section 22 calculates, as the amount of change δ of the blood glucose level G, a difference value between the blood glucose level GL at the point in time TS and the blood glucose level GH at the point in time TE of the peak after the point in time TS (S2). The sugar-content estimating section 23 specifies, using the reference information R stored in the storage device 13, the ingested sugar content M corresponding to the amount of change δ acquired by the information acquiring section 22 (S3). The sugar-content estimating section 23 causes the display device 14 to display the ingested sugar content M estimated by the procedure explained above (S4).

As explained above, in the first embodiment, since the ingested sugar content M by the subject is estimated according to the amount of change δ of the blood glucose level G of the subject, the subject does not need to input an amount of meal and meal content of the subject. Therefore, it is possible to reduce a burden of work necessary for grasping the ingested sugar content M. In the first embodiment, the reference information R representing the relation between the amount of change in the blood glucose level and the sugar content is generated according to the change in the blood glucose level of the subject at the time when the subject ingests the specific food. Therefore, it is possible to reduce the influence of an individual difference of the relation between the amount of change in the blood glucose level and the sugar content and highly accurately estimate the ingested sugar content M.

### Second Embodiment

A second embodiment of the present disclosure is explained. In the following illustrations, concerning components having the same functions as the functions of the components in the first embodiment, the reference numerals and signs used in the explanation of the first embodiment are applied. Detailed explanation of the components is omitted as appropriate.

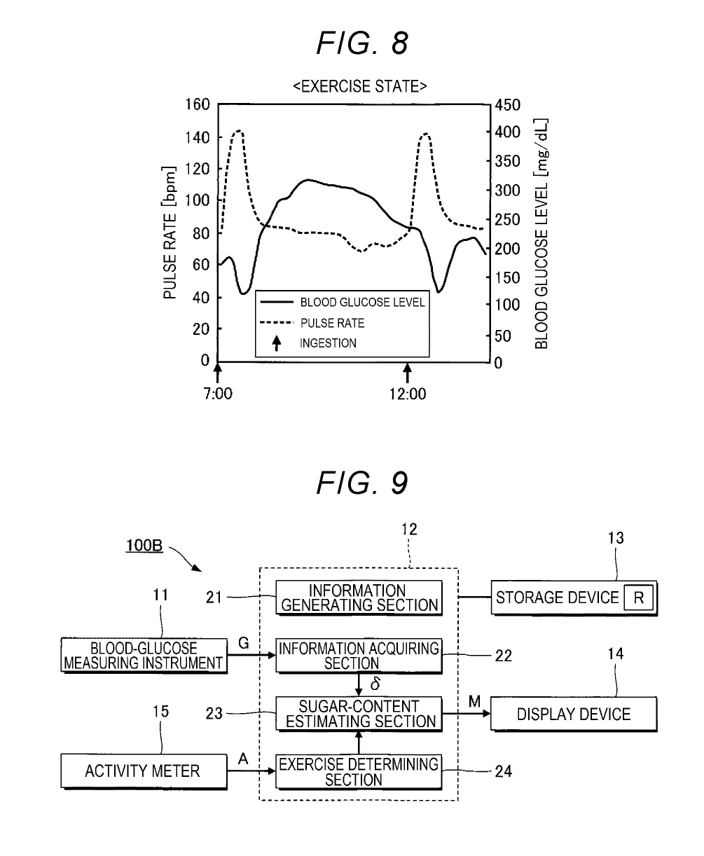

A rise in a blood glucose level after ingestion is affected by presence or absence of exercise immediately after the ingestion. In the second embodiment, the ingested sugar content M is estimated taking into account presence or absence of exercise of a subject. FIGS. 7 and 8 are graphs showing temporal changes of a blood glucose level and a pulse rate. In FIGS. 7 and 8, it is assumed that the subject ingests specific food at a plurality of points in time (7:00 and 12:00). FIG. 7 is a graph showing temporal changes in a blood glucose level and a pulse rate when the subject maintains a rest state immediately after ingestion. FIG. 8 is a graph showing temporal changes of a blood glucose level and a pulse rate when the subject exercises immediately after ingestion. A rise in the pulse rate by the exercise can be confirmed from FIG. 8.

As it is understood from the comparison of FIGS. 7 and 8, even when the same amount of sugar is ingested, there is a tendency that the rise in the blood glucose level is suppressed when the subject exercises immediately after the ingestion compared with when the subject maintains the rest state. Based on the tendency, in the second embodiment, the presence or absence of exercise by the subject is reflected on the estimation of the ingested sugar content M.

FIG. 9 is a block diagram illustrating the configuration of an organism analyzing apparatus 100B in the second embodiment. As illustrated in FIG. 9, the organism analyzing apparatus 100B in the second embodiment has a configuration in which an activity meter 15 is added to the organism analyzing apparatus 100A in the first embodiment. The activity meter 15 measures an activity amount A of the subject. The activity amount A is an indicator indicating a degree of activity of the subject. For example, a consumed calorie, the number of steps, and exercise intensity are illustrated as the activity amount A. The measurement of the activity amount A by the activity meter 15 is repeated, for example, at a predetermined cycle. The activity meter 15 separate from the organism analyzing apparatus 100B may be connected to the organism analyzing apparatus 100B by wire or radio. That is, the activity meter 15 is omitted from the organism analyzing apparatus 100B.

As illustrated in FIG. 9, the control device 12 in the second embodiment functions as an exercise determining section 24 in addition to the same components (the information generating section 21, the information acquiring section 22, and the sugar-content estimating section 23) as the components in the first embodiment. Functions of the information generating section 21 and the information acquiring section 22 are the same as the functions in the first embodiment.

The exercise determining section 24 determines presence or absence of exercise by the subject. Specifically, the exercise determining section 24 determines presence or absence of exercise by the subject according to the activity amount A measured by the activity meter 15. For example, when a state in which the activity amount A is larger than a predetermined threshold continues for a predetermined time, the exercise determining section 24 determines that the subject is exercising. The determination of presence or absence of exercise by the exercise determining section 24 is repeated, for example, at a predetermined cycle.

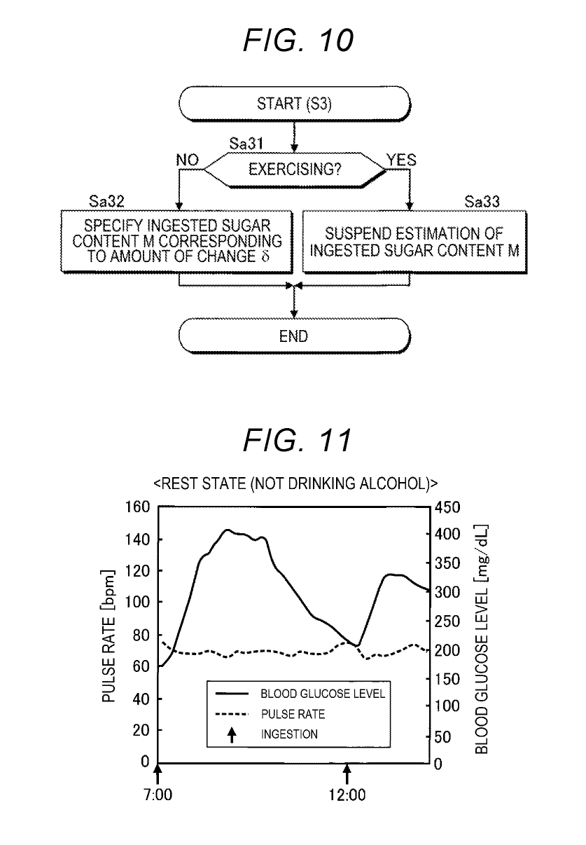

The sugar-content estimating section 23 estimates the ingested sugar content M of the subject according to a result of the determination by the exercise determining section 24 (i.e., the presence or absence of exercise by the subject). FIG. 10 is a flowchart illustrating an operation in which the sugar-content estimating section 23 in the second embodiment estimates the ingested sugar content M (S3). As illustrated in FIG. 10, when the exercise determining section 24 determines that the subject is not exercising (NO in Sa31), the sugar-content estimating section 23 estimates the ingested sugar content M corresponding to the amount of change δ of the blood glucose level G according to the same procedure as the procedure in the first embodiment (Sa32). An operation in which the sugar-content estimating section 23 causes the display device 14 to display the ingested sugar content M estimated by the sugar-content estimating section 23 is the same as the operation in the first embodiment. On the other hand, when the exercise determining section 24 determines that the subject is exercising (YES in Sa31), the sugar-content estimating section 23 suspends the estimation of the ingested sugar content M (Sa33). That is, the estimation of the ingested sugar content M is not executed.

In the second embodiment, the same effects as the effects in the first embodiment are realized. In the second embodiment, the ingested sugar content M is estimated according to the presence or absence of exercise by the subject. Therefore, there is an advantage that it is possible to highly accurately estimate the ingested sugar content M compared with a configuration in which the ingested sugar content M is estimated irrespective of the presence or absence of exercise.

In the illustration explained above, the execution and the suspension of the estimation of the ingested sugar content M is switched according to the presence or absence of exercise by the subject. However, a method of reflecting the presence or absence of exercise by the subject on the estimation of the ingested sugar content M is not limited to the illustration explained above.

For example, it is suitable to estimate the ingested sugar content M according to the amount of change δ of the blood glucose level G as in the second embodiment when the exercise determining section 24 determines that the subject is not exercising and correct the ingested sugar content M when the exercise determining section 24 determines that the subject is exercising. Specifically, the sugar-content estimating section 23 calculates the ingested sugar content M by multiplying initial ingested sugar content M0 specified from the amount of change δ of the blood glucose level G using the reference information R by a correction value α. The correction value α is controlled according to a degree of exercise of the subject. The correction value α is controlled according to the degree of the exercise of the subject. Specifically, a multiplied value of the activity amount A and an activity time is a suitable example of the correction value α. As the activity amount A of the subject is larger or the activity time is longer, the correction value α is set to a larger numerical value. That is, when the subject is exercising, the ingested sugar content M0 is corrected such that a decrease in the ingested sugar content M due to the exercise is compensated. As it is understood from the above explanation, processing for estimating the ingested sugar content M according to a result of the determination by the exercise determining section 24 includes processing for switching the execution and the suspension of the estimation of the ingested sugar content M according to the presence or absence of exercise and processing for correcting the ingested sugar content M0 according to the presence or absence of exercise.

### Third Embodiment

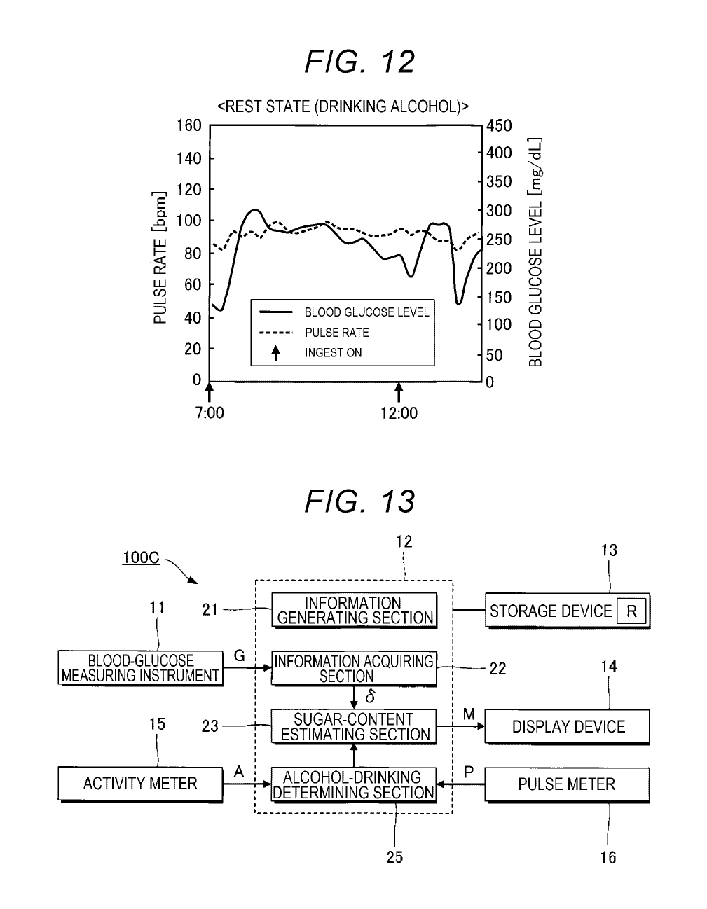

A rise in a blood glucose level after ingestion is affected by presence or absence of alcohol drinking (i.e., ingestion of alcohol) during the ingestion. In the third embodiment, the ingested sugar content M is estimated taking into account the presence or absence of alcohol drinking of a subject. FIGS. 11 and 12 are graphs showing temporal changes of a blood glucose level and a pulse rate when the subject ingests specific food at a plurality of points in time (7:00 and 12:00). FIG. 11 is a graph showing temporal changes of a blood glucose level and a pulse rate when the subject ingests food without drinking alcohol. FIG. 12 is a graph showing temporal changes of a blood glucose level and a pulse rate when the subject ingests food while drinking alcohol.

As it is understood from comparison of FIGS. 11 and 12, even when the same amount of sugar is ingested, there is a tendency that a rise in a blood glucose level is suppressed when the subject drinks alcohol simultaneously with food ingestion compared with when the subject does not drink alcohol. Based on the tendency, in the third embodiment, the presence or absence of alcohol drinking by the subject is reflected on the estimation of the ingested sugar content M.

FIG. 13 is a block diagram illustrating the configuration of an organism analyzing apparatus 100C in the third embodiment. As illustrated in FIG. 13, the organism analyzing apparatus 100C in the third embodiment has a configuration in which the activity meter 15 and a pulse meter 16 are added to the organism analyzing apparatus 100A in the first embodiment. As explained in the second embodiment, the activity meter 15 repeatedly measures the activity amount A of the subject.

The pulse meter 16 measures a pulse rate P of the subject. The pulse rate P is the number of times of pulses per unit time. A publicly-known technique is optionally adopted for the measurement of the pulse rate P. For example, it is suitable to adopt the pulse meter 16 configured to estimate the pulse rate P from a time sequence of intensity of light passed through the body of the subject and received by an optical sensor. The measurement of the pulse rate P by the pulse meter 16 is repeated, for example, at a predetermined cycle. The pulse meter 16 or the activity meter 15 separate from the organism analyzing apparatus 100C may be connected to the organism analyzing apparatus 100C by radio or wire. That is, the pulse meter 16 or the activity meter 15 is omitted from the organism analyzing apparatus 100C.

As illustrated in FIG. 13, the control device 12 in the second embodiment functions as an alcohol-drinking determining section 25 in addition to the same components (the information generating section 21, the information acquiring section 22, and the sugar-content estimating section 23) as the components in the first embodiment. Functions of the information generating section 21 and the information acquiring section 22 are the same as the functions in the first embodiment.

The alcohol-drinking determining section 25 determines presence or absence of alcohol drinking by the subject. Specifically, the alcohol-drinking determining section 25 determines presence or absence of alcohol drinking by the subject according to the activity amount A measured by the activity meter 15 and the pulse rate P measured by the pulse meter 16. The determination of presence or absence of alcohol drinking by the alcohol-drinking determining section 25 is repeated, for example, at a predetermined cycle.

When the activity amount A is smaller than a predetermined threshold, the subject is presumed to be in a rest state (a state in which the subject is not exercising). When the subject is in the rest state, usually, the pulse rate P is smaller than a predetermined threshold. However, the pulse rate P tends to be raised by alcohol drinking. Therefore, when the pulse rate P is larger than the threshold irrespective of the rest state of the subject, the subject is presumed to be drinking alcohol. Based on the tendency, when the activity amount A is smaller than the threshold and the pulse rate P is larger than the threshold, the alcohol-drinking determining section 25 determines that the subject is drinking alcohol. On the other hand, when the activity amount A is larger than the threshold or when the pulse rate P is smaller than the threshold, the alcohol-drinking determining section 25 determines that the subject is not drinking alcohol.

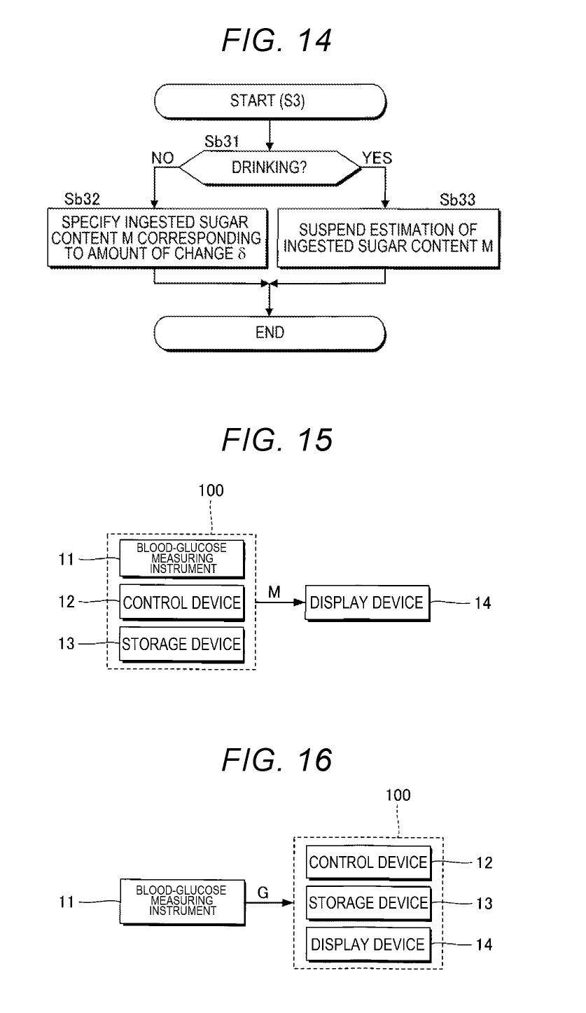

The sugar-content estimating section 23 estimates the ingested sugar content M of the subject according to a result of the determination by the alcohol-drinking determining section 25 (i.e., the presence or absence of alcohol drinking by the subject). FIG. 14 is a flowchart illustrating an operation in which the sugar-content estimating section 23 in the third embodiment estimates the ingested sugar content M (S3). As illustrated in FIG. 14, when the alcohol-drinking determining section 25 determines that the subject is not drinking alcohol (NO in Sb31), the sugar-content estimating section 23 estimates the ingested sugar content M corresponding to the amount of change δ of the blood glucose level G according to the same procedure as the procedure in the first embodiment (Sb32). An operation in which the sugar-content estimating section 23 causes the display device 14 to display the estimated ingested sugar content M is the same as the operation in the first embodiment. On the other hand, when the alcohol-drinking determining section 25 determines that the subject is drinking alcohol (YES in Sb31), the sugar-content estimating section 23 suspends the estimation of the ingested sugar content M (Sb33). That is, the estimation of the ingested sugar content M is not executed.

In the third embodiment, the same effects as the effects in the first embodiment are realized. In the third embodiment, since the ingested sugar content M is estimated according to the presence or absence of alcohol drinking by the subject, there is an advantage that it is possible to highly accurately estimate the ingested sugar content M compared with a configuration in which the ingested sugar content M is estimated irrespective of the presence or absence of alcohol drinking.

In the illustration explained above, the execution and the suspension of the estimation of the ingested sugar content M is switched according to the presence or absence of alcohol drinking by the subject. However, a method of reflecting the presence or absence of alcohol drinking by the subject on the estimation of the ingested sugar content M is not limited to the illustration explained above.

For example, it is suitable to estimate the ingested sugar content M according to the amount of change δ of the blood glucose level G as in the third embodiment when the alcohol-drinking determining section 25 determines that the subject is not drinking alcohol and correct the ingested sugar content M when the alcohol-drinking determining section 25 determines that the subject is drinking alcohol. Specifically, the sugar-content estimating section 23 calculates the ingested sugar content M by multiplying the initial ingested sugar content M0 specified from the amount of change δ of the blood glucose level G using the reference information R by a correction value β. That is, when the subject is drinking alcohol, the ingested sugar content M0 is corrected such that a decrease in the ingested sugar content M due to the alcohol drinking is compensated. As it is understood from the above explanation, processing for estimating the ingested sugar content M according to a result of the determination by the alcohol-drinking determining section 25 includes processing for switching the execution and the suspension of the estimation of the ingested sugar content M according to the presence or absence of alcohol drinking and processing for correcting the ingested sugar content M0 according to the presence or absence of alcohol drinking.

The configuration in the second embodiment for estimating the ingested sugar content M according to the presence or absence of exercise by the subject and the configuration in the third embodiment for estimating the ingested sugar content M according to the presence or absence of alcohol drinking by the subject may be combined. For example, the exercise determining section 24 that determines presence or absence of exercise by the subject according to the activity amount A measured by the activity meter 15 may be added to the third embodiment. When the exercise determining section 24 determines that the subject is exercising or when the alcohol-drinking determining section 25 determines that the subject is drinking alcohol, the sugar-content estimating section 23 suspends the estimation of the ingested sugar content M. In the configuration explained above, there is an advantage that it is possible to share the activity meter 15 for the determination by the exercise determining section 24 and the determination by the alcohol-drinking determining section 25.

### Modifications

The forms illustrated above can be variously modified. Modes of specific modifications that can be applied to the forms explained above are illustrated below. Two or more modes optionally selected from the following illustrations can be combined as appropriate in a range in which the modes are not contradictory to one another.

(1) In the forms explained above, the start point TS of ingestion is specified according to the time sequence of the blood glucose level G measured by the blood-glucose measuring instrument 11. However, a configuration and a method for specifying the start point TS of ingestion by the subject are not limited to the illustration explained above. For example, in a configuration in which the subject designates a start of ingestion to an organism analyzing apparatus 100 (100A, 100B, or 100C) through operation on an operation device (not illustrated), a point in time when the subject operates the operation device is specified as the start point TS of ingestion. According to the forms for specifying the start point TS of ingestion from the time sequence of the blood glucose level G, the subject does not need to designate a start of ingestion through operation on the operation device. Therefore, the effect that it is possible to reduce a burden of work necessary for estimating the ingested sugar content M is particularly conspicuous.

(2) In the second embodiment, the presence or absence of exercise by the subject is determined according to the activity amount A measured by the activity meter 15. However, a configuration and a method for determining presence or absence of exercise by the subject are not limited to the illustration explained above. For example, the exercise determining section 24 may determine presence or absence of exercise by the subject according to acceleration detected by an acceleration sensor worn by the subject. Considering a tendency that a pulse rate rises according to an activity of the subject, the exercise determining section 24 may determine presence or absence of exercise of the subject according to the pulse rate of the subject.

(3) In the third embodiment, the presence or absence of alcohol drinking by the subject is determined according to the activity amount A measured by the activity meter 15 and the pulse rate P measured by the pulse meter 16. However, a configuration and a method for determining presence or absence of alcohol drinking is not limited to the illustration explained above. For example, the presence or absence of alcohol drinking by the subject may be determined using a detector that measures alcohol concentration from exhaled air of the subject.

(4) In the forms explained above, the organism analyzing apparatus 100 including the display device 14 is illustrated. However, the display device 14 separate from the organizing analyzing apparatus 100 may be connected to the organism analyzing apparatus 100 by wire or radio. For example, as illustrated in FIG. 15, the blood-glucose measuring instrument 11, the control device 12, and the storage device 13 may be mounted on the organism analyzing apparatus 100. The ingested sugar content M may be displayed on the display device 14 separate from the organism analyzing apparatus 100. The display device 14 shown in FIG. 15 is mounted on an information terminal such as a cellular phone or a smartphone. As illustrated in FIG. 16, the control device 12, the storage device 13, and the display device 14 may be mounted on the organism analyzing apparatus 100. The blood glucose level G maybe transmitted from the blood-glucose measuring instrument 11 separate from the organism analyzing apparatus 100 to the organism analyzing apparatus 100. The organism analyzing apparatus 100 shown in FIG. 16 is realized by the information terminal such as the cellular phone or the smartphone.

(5) In the forms explained above, the ingested sugar content M estimated by the sugar-content estimating section 23 is displayed on the display device 14. However, information displayed on the display device 14 is not limited to the ingested sugar content M. For example, a diagram or a graph representing the time sequence of the ingested sugar content M may be displayed on the display device 14. A comment (proper/excessive/insufficient) corresponding to the ingested sugar content M may be displayed on the display device 14. In the illustration explained above, the information concerning the ingested sugar content M is displayed on the display device 14. However, the information concerning the ingested sugar content M may be notified to the subject with sound. It is also possible to assume a configuration for transmitting the information concerning the ingested sugar content M from a communication device to another communication device or a configuration for storing the information concerning the ingested sugar content M in a portable recording medium detachably attachable to the organism analyzing apparatus 100.

(6) A specific form of the organism analyzing apparatus 100 is optional. It is possible to realize an organism analyzing apparatus of any form such as a wristwatch type wearable on a wrist of the subject, a patch type stickable to the body of the subject, an ear-wearing type wearable on an ear of the subject, a finger-wearable type (e.g., a nail-wearable type) wearable on a fingertip of the subject, or a head-wearable type wearable on the head of the subject.

(7) As in the illustration explained above, the organism analyzing apparatus 100 according to the forms explained above is realized by cooperation of the control device 12 such as the CPU and the computer program. A computer program according to a preferred mode of the present disclosure can be provided in a form of storage in a computer-readable recording medium and installed in a computer. The computer program stored in a recording medium included in a distribution device can also be provided to a computer in a form of distribution via a communication network. The recording medium is, for example, a non-transitory recording medium. An optical recording medium (an optical disk) such as a CD-ROM is a good example. However, the recording medium can include a recording medium of publicly-known any form such as a semiconductor recording medium or a magnetic recording medium. The non-transitory recording medium includes any recording medium excluding a transitory propagating signal and does not exclude a volatile recording medium.

What is claimed is:

1. An organism analyzing apparatus comprising:
an information acquiring section configured to acquire a plurality of blood glucose levels in a time sequence measured by a blood-glucose measuring instrument and calculate, based on the time sequence of the blood glucose level, an amount of change in the blood glucose level due to ingestion of an organism;
a storage device configured to store reference information representing a relation between the amount of change in the blood glucose level and ingested sugar content; and
a sugar-content estimating section configured to estimate the ingested sugar content due to the ingestion using the amount of change in the blood glucose level and the reference information.

2. The organism analyzing apparatus according to claim 1, further comprising an alcohol-drinking determining section configured to determine presence or absence of alcohol drinking by the organism, wherein
the sugar-content estimating section estimates the sugar content according to a result of the determination by the alcohol-drinking determining section.

3. The organism analyzing apparatus according to claim 1, further comprising an exercise determining section connected by radio or wire and configured to determine, based on an activity amount of the organism output from an activity meter that measures the activity amount, presence or absence of exercise of the organism, wherein
the sugar-content estimating section estimates the sugar content according to a result of the determination by the exercise determining section.

4. The organism analyzing apparatus according to claim 3, further comprising an exercise determining section configured to determine presence or absence of exercise of the organism, wherein
the sugar-content estimating section estimates the sugar content according to a result of the determination by the exercise determining section.

5. The organism analyzing apparatus according to claim 1, wherein the information acquiring section determines a start point of the ingestion from the plurality of blood glucose levels and determines an amount of change in the blood glucose level based on the blood glucose level at the start point among the plurality of blood glucose levels.

6. The organism analyzing apparatus according to claim 2, wherein the information acquiring section determines a start point of the ingestion from the plurality of blood glucose levels and determines an amount of change in the blood glucose level based on the blood glucose level at the start point among the plurality of blood glucose levels.

7. The organism analyzing apparatus according to claim 3, wherein the information acquiring section determines a start point of the ingestion from the plurality of blood glucose levels and determines an amount of change in the blood glucose level based on the blood glucose level at the start point among the plurality of blood glucose levels.

8. The organism analyzing apparatus according to claim 4, wherein the information acquiring section determines a start point of the ingestion from the plurality of blood glucose levels and determines an amount of change in the blood glucose level based on the blood glucose level at the start point among the plurality of blood glucose levels.

9. A sugar-content estimating method conducted by a control device, comprising:
acquiring a plurality of blood glucose levels in a time sequence from a blood-glucose measuring instrument;
a control device calculating, based on the plurality of blood glucose levels, an amount of change in the blood glucose level due to ingestion of an organism; and
estimating, based on reference information representing a relation between the amount of change in the blood glucose level and ingested sugar content, ingested sugar content corresponding to the amount of change in the blood glucose level.

10. The sugar-content estimating method according to claim 9, further comprising determining presence or absence of alcohol drinking by the organism, wherein
the sugar content is estimated according to a result of the determination.

11. The sugar-content estimating method according to claim 9, further comprising determining, based on an activity amount of the organism output from an activity meter that is connected by wire or radio and measures the activity amount and a pulse rate of the organism output from a pulse meter that is connected by wire or radio and measures the pulse rate, presence or absence of alcohol drinking.

12. An organism analyzing apparatus comprising:
an activity meter configured to measure an activity amount of an organism;
a pulse meter configured to measure a pulse rate of the organism; and
a control device configured to determine, based on the activity amount and the pulse rate, presence or absence of alcohol drinking, calculate, based on a plurality of blood glucose levels acquired from a blood-glucose measuring instrument, an amount of change in the blood glucose level due to ingestion by the organism, and calculate ingested sugar content due to the ingestion using the presence or absence of alcohol drinking and the amount of change in the blood glucose level.

13. The organism analyzing apparatus according to claim 12, further comprising a storage device configured to store reference information representing a relation between the amount of change in the blood glucose level and the ingested sugar content, wherein
the control device calculates the ingested sugar content due to the ingestion using the presence or absence of alcohol drinking, the amount of change in the blood glucose level, and the reference information.

14. An organism analyzing apparatus comprising:
an activity meter configured to measure an activity amount of an organism;
a blood-glucose measuring instrument configured to measure a blood glucose level of the organism; and
a control device configured to determine, based on the activity amount, presence or absence of exercise, acquire a plurality of blood glucose levels in a time sequence from the blood-glucose measuring instrument, calculate, based on the plurality of blood glucose levels, an amount of change in the blood glucose level due to ingestion by the organism, and calculate ingested sugar content due to the ingestion using the presence or absence of exercise and the amount of change in the blood glucose level.

15. The organism analyzing apparatus according to claim 14, further comprising a storage device configured to store reference information representing a relation between the amount of change in the blood glucose level and the ingested sugar content, wherein
the control device calculates the ingested sugar content due to the ingestion using the presence or absence of alcohol drinking, the amount of change in the blood glucose level, and the reference information.

16. The organism analyzing apparatus according to claim 14, wherein the control device suspends the calculation of the ingested sugar content when determining based on the activity amount that the organism is exercising and executes the calculation of the ingested sugar content when determining that the organism is not exercising.

17. The organism analyzing apparatus according to claim 14, further comprising a pulse meter configured to measure a pulse rate of the organism, wherein
the control device determines, based on the activity amount and the pulse rate, presence of absence of alcohol drinking and calculates the ingested sugar content due to the ingestion using the presence or absence of alcohol drinking and the amount of change in the blood glucose level.

18. The organism analyzing apparatus according to claim 15, further comprising a pulse meter configured to measure a pulse rate of the organism, wherein
the control device determines, based on the activity amount and the pulse rate, presence of absence of alcohol drinking and calculates the ingested sugar content due to the ingestion using the presence or absence of alcohol drinking, the amount of change in the blood glucose level, and the reference information.
